(19) United States
(12) Patent Application Publication
(10) Pub. No.: US 2004/0087955 A1
(43) Pub. Date: May 6, 2004

(54) Tubular internal fixation for bone fractures and prostheses

(76) Inventor: Alessandro Bordi, Terni (IT)

Correspondence Address: Hedman & Costigan, 1185 Avenue of the Americas, New York, NY, 10036-2646, US

(21) Appl. No.: 10/470,512

(22) PCT Filed: Mar. 4, 2002
(86) PCT No.: PCT/IT02/00135

(30) Foreign Application Priority Data
Mar. 9, 2001 (IT) RM01A000123

(51) Int. Cl. A61B 17/56

(52) U.S. Cl. 606/74

(57) ABSTRACT

A device for the reduction and stabilisation of broken bone parts, such as those deriving from a fracture, that is particularly suitable for veterinary use. The device comprises a tubular band (1) as the main element, to be applied around the bone at the point of fracture or discontinuity, that is made of a metallic material and provided with a longitudinal aperture, equipped with multiple substantially pointed protrusions (2), distributed along its inner surface, for gripping the said broken bone parts. The tubular band (1) is sufficiently malleable to be wrapped tightly around the broken bone parts with the use of flexible cerclage elements, which are secured transversally around said tubular band and housed in external transverse grooves (3).

The present invention concerns a tubular internal fixation device for bone fractures and prostheses. More specifically, the invention relates to a device for reducing and stabilising broken bone fragments, particularly recommended for veterinary use, which comprises, as its main element, a rigid polycompressive band to be applied around the bone at the point of fracture or discontinuity.

As is known, both in human and veterinary medicine, the currently more widespread methods for reduction, containment and stabilization of bone fractures as an alternative or in addition to a closed technique of immobilization of the fractured part by casts or rigid bandaging with splints, are mainly of three types the application of metal plates or plaques held alongside and in strict contact with the fractured bone by connecting screws anchored in the bone itself the insertion of intramedullary rods or nails that longitudinally cross the two broken bone parts through the medullar canal, and external fixation devices. These are composed of a combination of transverse cross-bone rods or pins of which one or both ends remain outside the damaged section and are fixed to an external supporting frame, made up of one or more supporting longitudinal bars, possibly connected to each other by other external transverse elements.

It is also known that, although less traumatic as an initial treatment, a cast or immobilization using bandage with splints is often avoided—firstly, for the inconvenience resulting from a long immobilization of the damaged area such as muscular atrophy, that makes it necessary to have a long period of rehabilitation. Secondly, the use of casts involves hygiene and dermatological problems linked to the impossibility of exposing the skin under the cast to the air for the whole period necessary for healing fracture. Moreover, evidently, this system cannot be adopted if the fracture is accompanied by wounds or lacerations of the surrounding soft tissue. It is obvious that these inconveniences and contra-indications are even more critical with animals, if only for their incapacity to consciously cooperate in the treatment.

Internal devices composed of plates and screws obviously require surgery in which all the tissues must be cut in order to uncover the bone, and the screws fixing the metal plate up against the bone must perforate it, thereby introducing a weakening of the bone structure at all perforation points. The proper application of these devices allows keeping the parts of the fractured bone firmly locked to each other, thus increasing the stability of the fracture and accelerating the healing process of the bone tissues; it also places the patient in the condition to regain mobility almost immediately. However, these fixation systems are certainly more traumatic, as regards application, of the three previously listed solutions. This is also because the plates and screws, normally made of stainless steel for surgical applications, must be removed once the fractured bone has healed and this involves further surgery.

Intramedullary pins undoubtedly have the advantage of avoiding lesions to periosteal tissues and the soft tissues surrounding the bone, but they can only be used in certain types of fractures that are uncomplicated and with no comminuted fragments. However, in order to improve the resulting stability, they are often coupled with transverse screws anchored in the cortical area of the bone. Moreover, in veterinary applications, the main inconvenience lies in the fact that, since the animal feels it can make use of the fractured limb (and having a much higher pain threshold compared to man's), the animal's jumping will cause repeated compressions of the two broken parts and will gradually dislodge the pin.

As regards external fixation devices, although less traumatic, the treatment still requires placing pins or screws through the bone and thus perforating all the surrounding tissues. These devices are very much used in human orthopaedics and in many cases can reach such high levels of complexity and sophistication, above all in relation to the bars and other external supporting and regulating structures, that they are unsuitable in veterinary treatment, both for their cost and, above all, for problems of hygiene and device maintenance. Moreover, it is inevitable that the animal will feel discomfort from the external rigid element connected to its limb and will continually try to remove it.

Another more or less experienced inconvenience in all the aforesaid cases is the fact that, for fractures of a considerable magnitude, the duration of surgery, which always calls for drilled perforation of the bone in one or more places, may be quite lengthy, with a protracted exposure of the treated subject to anaesthesia.

Another internal fixation device that was widely used in the past but which is now in disuse, above all in human orthopaedics, is the so-called cerclage, in practice consisting of using orthopaedic wire to wire together fragments of bone in direct contact with the bone surface. On its own, this wiring technique can be applied, for example, in oblique fractures, while for straight transverse fractures the cerclage wiring technique should be combined with longitudinal metal plates going through the fractured area. One example of a device proposed for cerclage with orthopaedic wire is described in European patent application EP 0019062, in which the object of the invention concerns a mechanical device for holding and closely securing the ends of the wire around the bone.

The fundamental drawback for which cerclage is no longer used, at least in human treatment, consists of the fact that, because the wire is firmly secured around the bone (otherwise it would not fulfil its function of stabilising the fracture and of compressing the broken parts to each other), it seriously constrains periosteal blood supply. This leads to the autolysis of the bone tissues in the areas in contact with the wire and the resulting slackening of the cerclage, as well as a dangerous weakening of the bone in the area concerned. Another kind of cerclage, which still does not overcome the aforesaid drawback, is composed of a small flexible band that is tightly wrapped around the bone and secured by using an appropriate clamp or similar securing device. An example of this solution is described in the European patent application EP 0876798. Because this kind of band is completely smooth on the inside part that is in contact with the bone, not only does it still limit periosteal blood supply, but by slackening it will also move longitudinally along the bone on which it has been applied.

Within the band type devices used for bracing the bone in order to reduce and stabilize a fracture, the French patent FR 2211851 describes a clip for osteosynthesis that is particularly suitable for use with fractured ribs. The clip is composed of an elongated metal alloy sheet, the longer sides of which have some flexible peripheral tongues. These are hooked around the fractured rib and are bent round by using appropriate forceps for a tight fit. This solution obviously does not solve the problem of a close-knit contact with the bone that still compromises periosteal blood supply.

A very similar device to the previous one, but apparently designed for the fixation of fractures in long straight bones, is described in the European patent publication EP 0024635. In this case the sheet, made of steel or a similar metallic material, is equipped with a series of clasping tongues on the two longer sides and these wrap around the bone; however, they do not simply clasp the bone but also penetrate it with their sharp ends folded at right-angles in order to anchor tightly to it. It appears that the sheet does not grip the bone in all the area it covers so that the problem of periosteal blood supply seems to be partly overcome. However, the device only grips a little over half the external surface of the bone and the clasping elements are only found on the ends of the tongues, thus offering little guarantee of stability for the reduced fracture. Moreover, due to its design, the device needs a special instrument to push the clasping tongues into the bone and another instrument to slacken the sheet in order to remove it once the fracture has healed.

Still on the subject of band-shaped internal fixation devices, the U.S. Pat. No. 3,469,573 discloses a flat metal sheet band, largely of the kind referred to before, where the problem of periosteal blood supply is taken into account by equipping the inside of the band with a series of ribs across it (and longitudinal with respect to the bone on which it is then applied). This ribbing is carried out either by parallel folding of the metal sheet or with small bars made of another material (such as plastic) fixed on the inside of the band with appropriate pressure buttons. In this case, even if a cerclage is achieved that does not grasp the bone tightly along its entire perimeter, periosteal blood supply is still reduced to a certain extent because the internal protruding parts parallel to the bone are linear and continuous. Moreover, also due to the straight form of the internal protrusions, the possibility of dislodgement of the fixation by running along the bone itself cannot be ruled out. In any case, it must be borne in mind that the practical realisation of the device does not have the necessary simplicity to economically justify its use in the veterinary field.

Still more complex appears to be the device described in patent application EP 0295041, which also addresses the problem of stably immobilizing the broken bone parts without excessively hindering periosteal blood supply. This consists of an internal fixation device for fractures in long bones and is composed of a frame, in two or three longitudinal parts connected to each other in order to completely wrap around the fractured bone like a cage. The longitudinal bars and transverse connecting elements of the frame have, on the inside margins, rows of small teeth to bite into the bone surface in order to provide dynamic compression to the broken bone elements without there being a tight grip all around the bone surface. However, in this case, too, the inside protruding elements of the fixation device, that are distributed along mostly longitudinal continuous rows, tend to interfere to a certain extent with periosteal blood supply. Moreover, as is known, the complex realisation of the mentioned device, which also calls for the ad hoc production of a particular version for each diameter of bone to be treated, would not be economically viable in veterinary medicine.

On the basis of this previous state of the art, the present invention thus aims to provide an internal fixation device for bone fractures that is based on the cerclage principle, does not interfere with periosteal blood supply, and provides immediate and reliable stabilization of the fractured bone. In this way, the device reduces the risk of postoperative accidents, normally high in the case of animals, and is at the same time simple and economical to make and easy to apply through a much less surgically invasive treatment.

To this aim, the present invention proposes a polycompressive fixation device in the form of an incomplete or longitudinally open rigid band that braces the broken bone parts to be immobilised, appropriately adapting its shape to them, and is equipped with a number of internal unaligned and discontinuous protrusions in the form of punches, points or teeth. These are designed to grip the bone surface to be fixed by penetrating into it to a small degree and ensuring that the inside surface of the band itself does not adhere to the bone. The band is maintained stably wrapped around the broken bone parts by appropriate cerclage wiring that lies outside the device and does not rest on the bone. This configuration guarantees periosteal blood supply, thus favouring a quick healing process, provides a guide for bone callus and at the same time creates a stable immobilisation of the broken bone parts, allowing immediate mobility after the operation.

Thus, the present invention specifically provides an internal fixation device for bone fractures and prostheses comprising, as the main element, a longitudinally open tubular metal band suitable to be applied around broken bone parts to be fixed, and provided with multiple substantially point-shaped protrusions distributed along its internal surface and enabling a firm grip of the said broken bone parts, said tubular band being sufficiently flexible to adapt its shape to wrap around the said broken bone parts by varying the aperture of its longitudinal opening. The proposed fixation device also includes two or more flexible cerclage elements designed to wrap transversally around the said tubular band and are, preferably, composed of orthopaedic wire. These flexible elements are housed in specially designed transverse grooves—two or more in number, depending on band length and the characteristics of the fracture and of the broken bone parts—made on the outside surface of the band. The proximal and distal margins of the band itself are rounded so that they do not form sharp edges which could damage the soft tissue surrounding the bone or cause traumas by rubbing.

According to some specific embodiments of the invention, the substantially point-shaped protrusions provided on the inside surface of the tubular band may be composed of punchings carried out from outside the band itself, for example, of a conical shape, or perforated with four-pointed edges. The latter type are to be preferred because they provide greater stability in fixation by more firmly penetrating the bone. As an alternative, the protrusions may be created on the metal surface of the band by making drill-holes, where the in-jutting edges of the perforations act as a kind of uneven border around the perforation, which also penetrates and anchors into the surface layer of the bone.

If longer pointed protrusions are required on the inside surface of the metal band, these can be achieved, according to another series of embodiments of the invention, by screws, nails or similar pointed elements inserted from outside the tubular band. Here, too, the gripping ends of these elements can be conical or, preferably, have four points for a tighter grip on the bone. The embodiments using screws, nails or similar are particularly convenient to reduce and stabilise fractures in young animals which, because they are still growing, have expanding bone diameters during treatment. With the version of the polycompressive band using screws, nails or similar of a suitable length, in fact, the band can stay sufficiently detached from the bone to allow it to increase in diameter over time without interfering with the fixation device.

It must also be borne in mind that the device may be advantageously produced by using titanium instead of steel, either in a non-alloyed form or alloyed with small percentages of other metals. This material is particularly suitable for making surgical equipment and prostheses due to its greater lightness, at a parity of resistance, with respect to steel. Moreover, titanium is preferred above all for its very high biocompatibility, which makes a permanent osteointegrated implant possible, without needing to remove the device once the fracture has healed. Even the cerclage wires can also be, preferably, made of titanium instead of steel—either unalloyed titanium or alloyed with other metals.

For its very nature, the metal band of the present invention can adapt its shape to that of the bone section it is applied to. For example, once it is firmly secured, it may have a largely cylindrical shape, in order to adapt to the diaphysis of long bones, or a substantially truncated cone shape, to adapt to the epiphysis of long bones. Or, again, the band may have a substantially curved shape, adapting to the diaphysis of long bones with a non-perfectly linear shape, not infrequent in animals.

Further constructional and functional features of the internal fixation device of the present invention, as well as the advantageous aspects of its application, will be more evident with reference to some of its specific embodiments, illustrated by way of example in the attached figures, where:

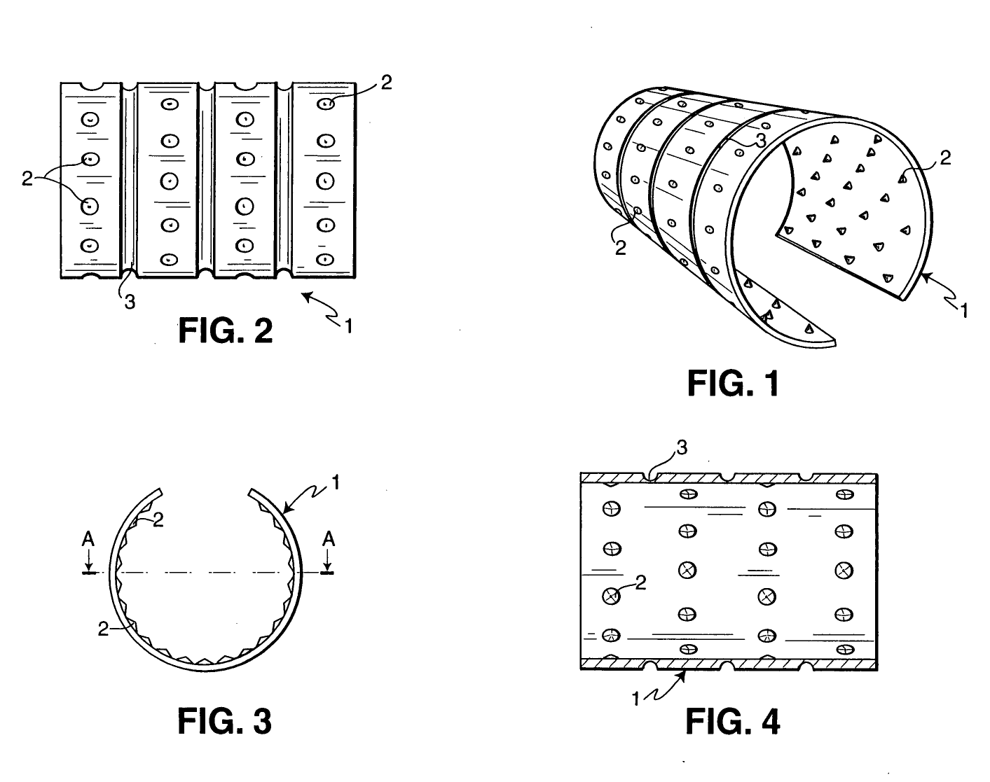

FIG. 1 is a perspective view of a first embodiment of the main band element of the proposed internal fixation device;

FIG. 2 is a front view of the same element shown in FIG. 1;

FIG. 3 is a side view of the same element shown in FIG. 1;

FIG. 4 is a longitudinal sectional view of the same element, taken along the plane A-A of FIG. 3;

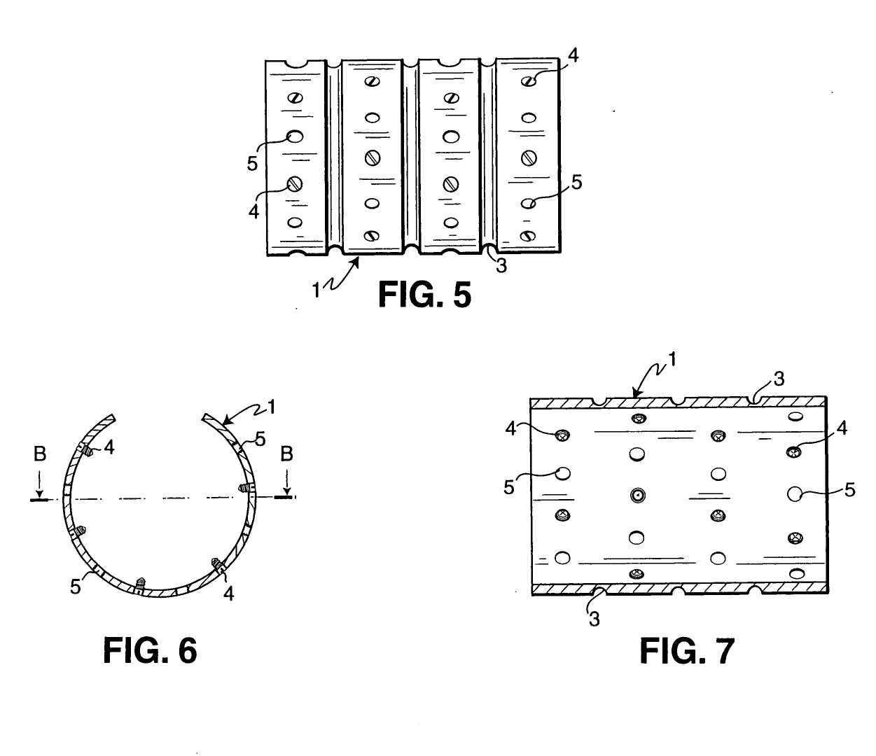

FIG. 5 is a front view of a second embodiment of the main band element of the proposed internal fixation device;

FIG. 6 is a cross-sectional view of the same element shown in FIG. 5; and

FIG. 7 is a longitudinal cross-section of the same element, taken along the plane B-B of FIG. 6.

As FIGS. 1-4 show, a particularly effective version of the internal fixation device according to the present invention includes, as main element, a tubular cylindrical band (1), open along one longitudinal side. This is made by using a titanium tube element of sufficient thickness to give the fixation device the necessary rigidity, but not so much to make the device too heavy or to make it difficult for the metal band to adapt its shape to the external shapes of the broken bone parts it is applied to. If it is applied to fractures on the diaphysis of long substantially straight bones, the tubular band (1) will largely maintain the shape shown in the figures, except for the fact that it may be wrapped around broken bone parts, varying the gap in its longitudinal opening in order to more firmly grip them via the punched protrusions (2). Given the possibility of changing the diameter of the tubular band (1), the same device can be adapted to various dimensions of bone to be immobilised, thus limiting the range of versions to have available when needing to carry out a quick operation, without having to produce an ad hoc piece according to the fracture to be treated.

Once it is applied to two broken bone parts of a fracture, in the course of an operation in which the necessary incisions have been made in the skin and in the soft tissue surrounding the bone concerned, the tubular band (1) is secured by tightening two or more cerclage wires (not shown) around it. The wires are housed in the specially designed transverse grooves (3) made in the outer surface of the tubular band (1). It is important to note that, unlike what was used with conventional cerclage, the wires in this case are not fastened around the bone but around the tubular band (1), which rests on the bone only on the points of the punched protrusions (2). The wires can be fastened without using complex and cumbersome securing clips such as the ones proposed in the prior art: the said clips not only took up space, but also risked damaging the surrounding soft tissue. In a much simpler manner, the ends of the wire are rolled up close to the longitudinal opening of the tubular band (1) and then they are folded, when possible, towards the inside of the band and tucked away in the gap of the tubular band (1).

In the creation of a device according to the present invention starting from a tubular element made of titanium or an alloy of titanium (for example, the alloy Ti-6Al-4V for prostheses and surgical applications, that contains about 6% of aluminium and about 4% of vanadium), the tubular element is first cut to size along its length, opened lengthwise and then grooved (3) on a lathe and punched (2) using a suitably shaped punching tool. As already noted, suitable protrusions can also be made on the inner surface of the tubular band (1) by drilling holes that have in-jutting metal edges.

Even if this is not shown in the figures, all edges of the tubular band (1) are not left sharp, but are preferably rounded off so that they do not damage the soft tissue that will surround the implanted tubular band (1).

The device according to the present invention is adaptable to the bone to be treated and is available in the already discussed variations of a largely cylindrical, truncated-conical or curved shape. It can be used not only for the reduction and stable fixation of simple fractures, but also for similar operations on multiple fractures and/or in the presence of bone material destruction caused by, for example, crushing, gunfire wounds and strong violent traumas. In these cases, some of the bone may be missing and the device according to the present invention will stabilise the two broken bone elements maintaining the physiological distances and optimally guiding the growth of bone callus. Moreover, the same device can also be used in cases of bone malformations, when it becomes necessary to remove part of the bone following abnormal growth or in the presence of pathologies such as bone tumours, where the tubular band can be easily used in order to replace the amputated parts. Finally, the proposed tubular band may also be an integral part of articular prostheses, such as hip prostheses, instead of the infibula and cements currently used to keep prostheses in place. The latter materials, in fact, besides being invasive, also have the inconvenience of wearing over time. In this specific case, the use of the tubular band of the present invention allows bone callus growth inside the band and thus affords a more reliable and permanent stabilisation of the prosthetic implant.

As is known, in veterinary medicine the treatment of bone fractures is mainly carried out for small animals. Large animals, such as adult cattle and horses, that have a fracture are normally put down due to the difficulties in applying the known methods to these large heavy creatures. The device according to the present invention can also be used with large animals, due to its ease and speed of application and considering that, if made in the appropriate material, it must not be removed later; all this along with the fact that it is also an economical treatment means that there is no need to put down large animals in case of bone fractures.

Another series of embodiments of the device according to the present invention that is suitable for particularly advantageous applications is shown in FIGS. 5-7. Here, the protrusions in the tubular band (1) are provided by screws (4), which are inserted in the holes (5) made in the tubular band (1). Indeed, the latter has elements corresponding to those of the device shown in FIGS. 1-4, including the rounded-off edges (not shown), and transverse grooves (3) for housing the cerclage wires (not shown). It is obvious that creating the protrusions with screws, nails or similarly pointed elements affords maximum flexibility in the use of the device, allowing, for example, the calibration of the length and the number and distribution of screws (4) as required. In the version shown in FIGS. 5-7, in particular, only a few screws (4) have been inserted in the holes (5), while other holes (5) have not been used. Moreover, the length of the screws (4) is such that they stand out only a little way from the inner surface of the tubular band (1), thus providing a kind of anchorage on the bone similar to the one created with the punched protrusions (2) shown in FIGS. 1-4.

In particular, this type of realisation may be advantageously used in the treatment of young animals for which a non-negligible growth in the bone concerned is to be expected. In this case, it is possible to use a tubular band (1) of a greater diameter than the one required at the time of the surgical operation, but with longer screws (4). In this way, the points of the screws (4) will still enable the device to firmly grip the broken bone elements to be immobilised, thus providing the necessary stability to the bone concerned, but at the same time allowing the normal growth of the animal without the tubular band (1) interfering with new bone formation. As the animal grows bigger, the screws (4) will become embedded in the new bone formation and the structure will present a complete osteointegration.

Compared to the solutions envisaged by the previous state of the art for the reduction, containment and stabilisation of bone fractures, or for the reconstruction of missing bone parts or the implanting of articular prostheses, the internal fixation device according to the present invention offers the advantage of considerable ease and speed of application, perfect bone reduction and stability, with the possibility of immediate mobility after the operation, a practically negligible interference with periosteal blood supply, as well as the capacity to reduce postoperative accidents because the device can stand up to torsion, longitudinal compression, stretching and bending. Moreover, as shown, when made of titanium or titanium alloy, the proposed internal fixation device is osteocompatible and lightweight, and can be left permanently in place. Finally, it can be adapted to any long bone by appropriately changing its shape quickly and immediately before the operation.

The present invention has been disclosed with particular reference to some specific embodiments thereof, but it should be understood that modifications and changes may be made by the persons skilled in the art without departing from the scope of the invention as defined in the appended claims.

1. An internal fixation device for bone fractures and prostheses comprising, as the main element, a longitudinally open tubular band (1), made of a metallic material, suitable to be applied around broken bone parts to be fixed, and provided with multiple substantially point-shaped protrusions (2, 4) distributed along its inner surface and enabling a firm grip of the said broken bone parts, said tubular band (1) being sufficiently flexible to adapt its shape to wrap around the said broken bone parts by varying the aperture of its longitudinal opening, said device also including two or more flexible cerclage elements that can be wrapped transversally around the said tubular band (1).

2. An internal fixation device according to claim 1, wherein said flexible cerclage elements are orthopaedic wires.

3. An internal fixation device according to claim 2, wherein said tubular band (1) is equipped with two or more transverse external grooves (3) that house the said cerclage wires.

4. An internal fixation device according to claims 1-3, wherein said substantially point-shaped protrusions are composed of punchings (2) carried out from the outside of the said tubular band (1) made of a metallic material.

5. An internal fixation device according to claim 4, wherein said punchings (2) are of a conical shape.

6. An internal fixation device according to claim 4, wherein said punchings (2) are cut into four-pointed edges.

7. An internal fixation device according to claims 1-3, wherein said substantially point-shaped protrusions are made of the in-jutting edges of holes drilled from the outside.

8. An internal fixation device according to claims 1-3, wherein said substantially point-shaped protrusions consist of screws (4), nails or similar pointed elements inserted from the outside into the said tubular band (1).

9. An internal fixation device according to claim 8, wherein said screws (4), nails or similar elements have a conical pointed tip.

10. An internal fixation device according to claim 8, wherein said screws (4), nails or similar elements have four-pointed tips.

11. An internal fixation device according to claims 1-10, wherein said metallic material of the tubular band (1) is either unalloyed titanium or a titanium alloy for orthopaedic use.

12. An internal fixation device according to claims 2-11, wherein said orthopaedic wires are made of steel, unalloyed titanium or a titanium alloy.

13. An internal fixation device according to claims 1-12, wherein said tubular band (1), once secured around the said broken bone parts, has a substantially cylindrical shape, adapting itself to the diaphysis of long bones.

14. An internal fixation device according to claims 1-12, wherein said tubular band (1), once secured around the said broken bone parts, has a substantially truncated-cone shape, adapting itself to the epiphysis of long bones.

15. An internal fixation device according to claims 1-12, wherein said tubular band (1), once secured around the said broken bone parts, has a substantially curved shape, adapting itself to the diaphysis of long bones with a non-perfectly linear shape.
